(19) United States
(12) Patent Application Publication
(10) Pub. No.: US 2012/0330194 A1
(43) Pub. Date: Dec. 27, 2012

(54) APPARATUS AND METHOD FOR TREATING TISSUE WITH ULTRASOUND

(76) Inventors: Alexander Britva, Migdal Ha'emek (IL); Ziv Karni, Kfar Shmaryahu (IL); Yevgeny Pens, Haifa (IL)

(21) Appl. No.: 13/476,780

(22) Filed: May 21, 2012

Related U.S. Application Data
(60) Provisional application No. 61/487,732, filed on May 19, 2011.

(51) Int. Cl. A61N 7/00

(52) U.S. Cl. 601/2

(57) ABSTRACT

A treatment apparatus and method are disclosed for applying mechanical ultrasonic vibrations to an area of tissue. The apparatus comprises an applicator movable by an operator over the area of tissue to be treated. Mounted within the applicator are an ultrasonic transducer for generating mechanical vibrations and a sonotrode for transmitting the vibrations generated by the transducer to the tissue to be treated. The apparatus further comprises a sensor for measuring an operating parameter indicative of the efficiency of the acoustic coupling between the sonotrode and the tissue to be treated, and electronic circuitry for generating a signal (e.g. an alarm signal) when the efficiency of the acoustic coupling drops below a threshold.

### CROSS REFERENCE TO EARLIER APPLICATION

The present invention is based on and claims priority from U.S. Provisional Patent Application No. 61/487,732 which was filed by the Applicants on 19 May 2011.

### FIELD

The present invention relates to apparatus for treating tissue with ultrasound. In the present disclosure, “ultrasound” and “ultrasonic” refers to mechanical waves or vibrations at a frequency inaudible to humans, being at least 20 kHz, but preferably in a range from 50 to 200 kHz.

### BACKGROUND

A treatment apparatus and method have been disclosed to treat adipose tissue by application of ultrasonic mechanical vibrations. The apparatus comprises an applicator movable by an operator over the area of tissue to be treated, the applicator having mounted therein an ultrasonic transducer for generating mechanical vibrations and a sonotrode for transmitting the vibrations generated by the transducer to the tissue to be treated. Such an apparatus, and a method of carrying out such treatment, are described in WO2009/095894, which is herein incorporated by reference in its entirety, as if fully set forth herein. The apparatus may be used to treat adipocytes in any location of the body, including but not limited to the abdominal region, the face, the back, the calves, the buttocks and the thighs.

As described in WO2009/095894, but without wishing to be limited by a single hypothesis, the apparatus operates by selectively damaging adipose tissue beneath the surface of the skin by delivering transverse ultrasound waves to the adipose tissue via the skin surface. Such delivery of ultrasound waves occurs through acoustic coupling of the device's sonotrode to the skin, as described in greater detail below. The transverse ultrasound waves penetrate and propagate to the fibers/membrane structure of adipose tissue to deform and damage adipocyte cell membranes by repeatedly stretching and allowing to relax the cell membranes, while causing substantially no collateral damage to surrounding tissue.

As is known, ultrasonic vibrations can propagate either as longitudinal waves or transverse waves. In longitudinal vibrations, also referred to as pressure waves, particles vibrate parallel to the direction of wave propagation. In transverse vibrations, also known as shear waves, particle motion is perpendicular to the direction of wave propagation. In embodiments of the present invention, transverse ultrasonic vibrations may be applied either alone or in combination with longitudinal vibrations. This may be carried out using a device that is configured to deliver both longitudinal and transverse mechanical vibrations at ultrasonic frequency. Such a device may use a mushroom-shaped sonotrode having a proximal portion acoustically coupled to the transducer, and a narrower neck portion terminating in a distal portion that is wider than the neck portion. Such a sonotrode may be configured to provide a mode where at least 30%, by energy, of the induced ultrasound vibrations within the distal portion of the sonotrode are transverse ultrasound vibrations in a direction that is substantially perpendicular to the neck axis, within a tolerance of 20°.

### SUMMARY OF EMBODIMENTS

Embodiments of the present invention relate to an ultrasound treatment method comprising: operating an ultrasound device to transmit ultrasound vibrations including transverse ultrasound vibrations to biological tissue via a device sonotrode in contact therewith; monitoring an ultrasound device operating parameter that is descriptive of a coupling efficiency between the sonotrode and the biological tissue; and contingent upon the monitored coupling efficiency being below a threshold value, generating a coupling alert signal.

In one non-limiting example, an operator manually treats administers ultrasound energy to a patient using a sonotrode coupled to and/or in contact with the patient's skin. In this example, transverse mechanical waves of an ultrasound frequency are delivered to the tissue beneath the skin—for example, to induce fat reduction and/or damage adipocytes therein.

As the applicator is moved over the skin surface, the quality of the coupling between the sonotrode and the patient's skin may fluctuate. In response to sensing of a poor coupling therebetween, the generated alert signal may alert the operator of a poor coupling between the sonotrode and the biological tissue. Conversely, when the operator remedies the situation (for example, by applying more pressure or reorienting the sonotrode or employing better gel), the coupling frequency may increase, and the alert signal may cease.

Some embodiments relate to a multi-mode device including: (i) a “cold” or “transverse” operating mode where ultrasound energy delivered to the patient is primarily energy of transverse ultrasound waves, and (ii) a “hot” or “longitudinal” operating mode where ultrasound energy delivered to the patient is primarily energy of longitudinal ultrasound waves. Such is a device is disclosed in WO2009/095894.

In some embodiments, the ultrasound device may provide the transverse and longitudinal modes as follows: (i) when the device is in the longitudinal operating mode, ultrasound vibrations of an energy delivery surface facing and coupled to the biological tissue are primarily longitudinal ultrasound vibrations and (ii) when the device in the transverse operating mode, ultrasound vibrations of the energy delivery are primarily transverse ultrasound vibrations.

In some embodiments, it is possible to operate the device in the longitudinal mode (e.g. for a relatively brief period of time—e.g. at most 1 second or at most 0.5 seconds) in order conduct a longitudinal-mode measurement of the operating parameter (i.e. measurements carried out when the ultrasound device is in the longitudinal mode). Subsequently, when operating in the transverse operating mode, instead of conducting on a longitudinal-mode measurement of the operating parameter and/or instead of relying (or overly relying) on this measurement, it is possible to rely primarily on the previous longitudinal-mode measurement of the operating parameter. Thus, in some embodiments, the alert signal generated during transverse mode operation is based primarily upon the previous longitudinal-mode measurement of the operating parameter.

Towards this end, it may be advantageous to cycle between the two operating modes. As noted in the previous paragraph, it may not be necessary to operate the device in the longitudinal mode for an extended period of time, but only for a sufficient duration to acquire a reasonable longitudinal-mode measurement of the coupling efficient-descriptive operating parameter. Immediately thereafter, it is possible to operate the device in the transverse operating mode. Each cycle includes a longitudinal-mode portion when the device operates in the longitudinal mode followed by a traverse-mode portion when the device operates in the transverse mode. When the device, once again, operates in the longitudinal-mode, this defines the beginning of a new device cycle. This may be repeated any number of times (e.g. at least 5, at least 10, at least 20, at least 50, or at least 100).

It is now disclosed that relying on the longitudinal mode measurement of the operating parameter may obviate the need to rely on a more current but potentially less reliable traverse mode measurement of the coupling efficiency when regulating or generating an alert signal.

In some embodiments, it is possible to repeatedly operate the device longitudinal mode for a brief period of time (e.g. at most 2 seconds or at most 1 second or at most 0.5 seconds) in order to measure the operating parameter before subsequently transitioning to the transverse operating mode where deeper-penetrating transverse waves are administered. By alternating between operating modes, a device cycle (i.e. including a longitudinal-mode portion and a transverse-mode portion following immediately thereafter) is defined.

In various embodiments, the device cycle may be relatively rapid—for example, at least 0.2 Hz or at least 0.3 Hz or at least 0.4 Hz or at least 0.5 Hz or at least 0.75 Hz or at least 1 Hz. The device may thus cycle between the operating modes for any number of device cycles—e.g. at least two or at least five or at least ten or at least twenty device cycles.

In some embodiments, the sonotrode vibrates in the transverse mode for a substantial majority of each of the device cycles. In the present disclosure, a ‘substantial majority’ may be at least 60% or at least 70% or at least 75% or at least 80% or at least 90%.

The alert signal may be audio signal, a visual signal, or any combination thereof.

A number of techniques for sensing a coupling efficiency between the sonotrode and the biological tissue are disclosed herein. In one example, it is possible to sense a strength of vibrations reflected at an interface between the sonotrode and the tissue to be treated—for example, the reflected vibrations may set up a standing wave in the sonotrode of which the amplitude decreases with increase in coupling efficiency.

Alternatively or additionally, if the ultrasound device includes a transducer powered by an oscillatory drive circuit, and it is possible to measure a magnitude of the current drawn therefrom. When the sonotrode is tightly coupled to the biological tissue, the transfer of ultrasound energy thereto may be more efficient, allowing the transducer to consume current at a greater level.

Other coupling efficiency-descriptive operating parameters are disclosed below.

In some embodiments, in addition to sensing the operating parameter indicative of coupling efficiency, and in addition to the generating of the coupling alert signal, it is possible to sense one or more additional operating parameters, and to generate one or more respective additional alarm signals in accordance with the results of the sensing of the additional operating parameter(s).

For example, it may be possible to sense a speed of the applicator over a surface of the biological tissue, and to generate a motion alert signal when the speed or acceleration lies outside a desired range

Alternatively or additionally, it may be possible to sense a tilt of the sonotrode relative to a surface of the biological tissue and to generate an inclination alert signal when the inclination is outside a desired range

The additional alarm signals may, for example, be provided as visual or audio alarm signals.

Some embodiments relate to a treatment apparatus for applying mechanical ultrasonic vibrations to an area of tissue, comprising an applicator movable by an operator over the area of tissue to be treated, the applicator having mounted therein an ultrasonic transducer for generating mechanical vibrations and a sonotrode for transmitting the vibrations generated by the transducer to the tissue to be treated, the apparatus further comprising a sensor for measuring an operating parameter indicative of the efficiency of the acoustic coupling between the sonotrode and the tissue to be treated, and a processor for generating a signal when the efficiency of the acoustic coupling drops below a predetermined threshold.

In some embodiments, the sensor is responsive to vibrations reflected at an interface between the sonotrode and the tissue to be treated, the reflected vibrations setting up a standing wave in the sonotrode of which the amplitude decreases with increase in coupling efficiency. In such embodiments, the sensor may comprise at least one vibration sensor in contact with a region of the sonotrode close to an internode of the standing wave set up by the vibrations reflected at the interface between the sonotrode and the tissue to be treated.

In some embodiment, in which the transducer is powered by an oscillatory drive circuit, the operating parameter indicative of the efficiency of the acoustic coupling between the sonotrode and the tissue to be treated is the voltage developed across the transducer by the drive circuit.

In alternative embodiments, the operating parameter indicative of the efficiency of the acoustic coupling between the sonotrode and the tissue to be treated may be the current drawn by the transducer from the drive circuit or the Q-factor of the drive circuit.

The transducer generating the ultrasonic vibrations may be of any suitable type, such as a piezoelectric transducer, a magnetostrictive transducer or an electromagnetic acoustic transducer.

It is advantageous in some embodiments of the invention to provide further sensors to warn the operator of such other factors as incorrect speed, acceleration and inclination of the applicator that would affect the efficacy of the treatment.

The signal generated by the processor may coupled to a device for providing at least one of audible, visual and sensory feedback to a human operator to warn of incorrect operation of the apparatus or if the applicator is operated automatically then the signal may be used to vary the applied pressure, inclination or the speed of movement of the applicator.

In accordance with a second aspect of the invention, there is provided a treatment apparatus for applying mechanical ultrasonic vibrations to an area of tissue, comprising an applicator movable by an operator over the area of tissue to be treated, the applicator having mounted therein an ultrasonic transducer for generating mechanical vibrations and a sonotrode for transmitting the vibrations generated by the transducer to the tissue to be treated, wherein the transducer is operative to apply two different frequencies alternately to the sonotrode so as to cause the sonotrode to vibrate alternately in a longitudinal mode and in a transverse mode, the apparatus further comprising a sensor for measuring, at least during operation in the longitudinal mode, an operating parameter indicative of the efficiency of the acoustic coupling between the sonotrode and the tissue to be treated, and a processor for generating a signal when the efficiency of the acoustic coupling drops below a predetermined threshold.

### BRIEF DESCRIPTION OF THE DRAWINGS

The invention will now be described further, by way of example, with reference to the accompanying drawings, in which:

FIG. 1 is a perspective view of an applicator with part of its housing removed to expose the internal components,

FIG. 2A is a schematic section through the ultrasound transducer and sonotrode in FIG. 1,

FIG. 2B is a diagram showing the effect of changes in acoustic coupling standing waves set up in the sonotrode by reflection at the interface between the sonotrode and the tissue being treated,

FIG. 2C is a schematic diagram of the resonant circuit of the transducer,

FIG. 2D shows a detail of the distal end of the sonotrode contacting the tissue and establishing poor acoustic coupling,

FIG. 2E is a similar diagram to FIG. 2D, showing the distal end of the sonotrode contacting the tissue when establishing good acoustic coupling,

FIG. 3A is a diagram of the transducer and sonotrode of FIG. 1, showing the positions of vibrations sensors to detect the amplitude of the standing wave set up by acoustic reflection,

FIG. 3B is a diagram showing the amplitude of vibration created by the standing wave at different positions along the transducer and sonotrode of FIG. 3A,

FIG. 4 is a circuit diagram of a circuit for driving the transducer,

FIG. 5 is a block diagram of the entire treatment apparatus,

FIG. 6 is a block diagram of the ultrasound generator,

FIG. 7 is a flow chart of an algorithm for determining the quality of the acoustic coupling, and

FIG. 8 is a chart detailing the steps performed during operation of the treatment apparatus.

### DETAILED DESCRIPTION OF EMBODIMENTS

With specific reference now to the drawings in detail, it is stressed that the particulars shown are by way of example and for purposes of illustrative discussion of the preferred embodiments of the exemplary system only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

In the present disclosure ‘electronic circuitry’ is intended broadly to describe any combination of hardware, software and/or firmware.

Electronic circuitry may include may include any executable code module (i.e. stored on a computer-readable medium) and/or firmware and/or analog or digital electronic hardware element(s) including but not limited to field programmable logic array (FPLA) element(s), hard-wired logic element(s), microprocesor(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture. Electronic circuitry may be located in a single location or distributed among a plurality of locations where various circuitry elements may be in wired or wireless electronic communication with each other.

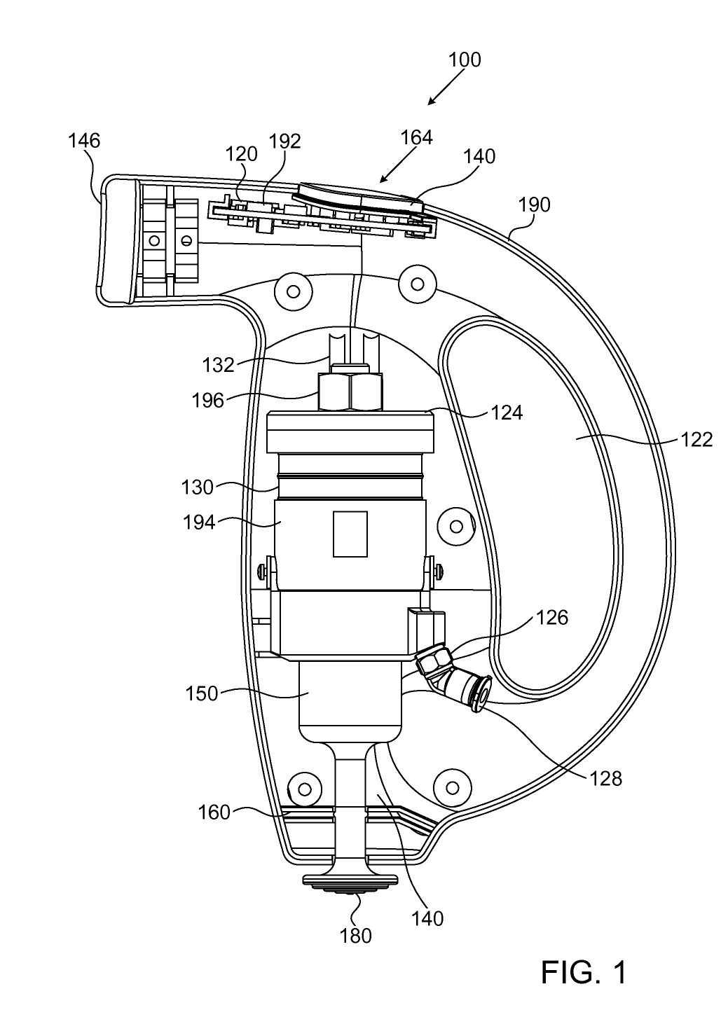

FIG. 1 shows an illustrative, exemplary non-limiting apparatus for ultrasound treatment according to at least some embodiments of the present invention. As shown, a treatment apparatus comprises an applicator 100 delivers ultrasound energy to biological tissue (not shown). The applicator 100 includes: (i) an ultrasound transducer 130 (for example, a piezo-ceramic transducer or a magnetostrictive-type ultrasound transducer or a transducer of any other type) for producing ultrasound vibrations at one or more frequencies; and (ii) a sonotrode 140 configured to deliver ultrasound energy (i.e., transverse or shear mechanical waves of an ultrasound frequency and optionally longitudinal ultrasound waves) provided by ultrasound transducer 130 to the biological tissue via an energy delivery surface 180 in contact with the biological tissue.

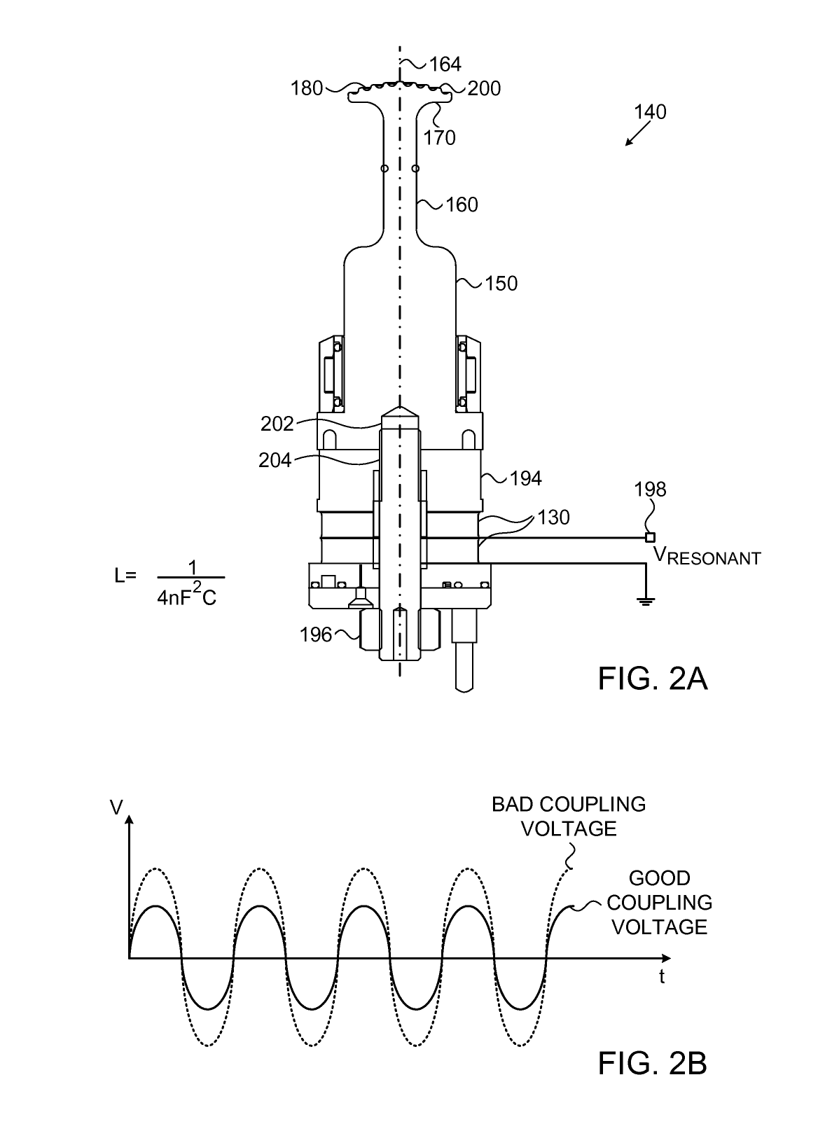

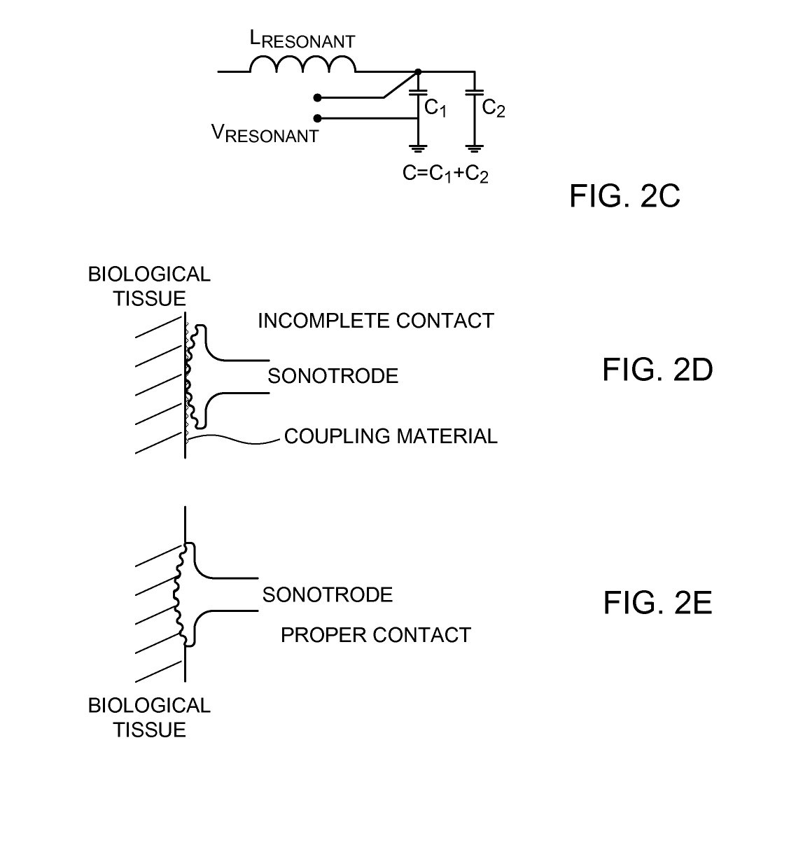

As shown in FIG. 2C the piezoelectric crystals constituting the transducer 130 behave as capacitors and can be connected in series or parallel with an inductor to form a resonant circuit. Like any solid object, the assembly of the sonotrode and the transducer will resonate at certain frequencies. The sonotrode 140 can furthermore vibrate in longitudinal and transverse modes and the frequency at which resonance occurs is dependent upon the mode of vibration. Thus, by varying the inductance in the resonant circuit of the transducer, the sonotrode can be excited in different modes.

For the treatment of adipocytes, it has been found that the transverse mode of vibration, during which the sonotrode is cold to the touch, is the more effective. Longitudinal excitation of transducer 130, during which the sonotrode is hot to the touch, is not typically used in the art of adipocyte treatment but is used in some embodiments of the invention in conjunction with the transverse mode, to enable acoustic coupling to be more accurately determined, for example for providing feedback to the operator.

In some embodiments, therefore, the treatment apparatus is a multi-mode device that is configured to be excited, alternately, in a cold mode, at a first frequency to deliver primarily transverse ultrasound energy to biological tissue and in a hot mode, at a second frequency to deliver primarily longitudinal transverse energy to the biological tissue. The frequency of alternation between the two modes is selected so that the acoustic coupling does not have time to change significantly between the two different modes. The frequency of alternation may range from several times per second to once every few seconds. Typically, the apparatus may be operated in the hot mode for between 10% and 25% of the time.

As shown, sonotrode 140, which may be constructed of a suitable material such as aluminum, is optionally mushroom-shaped and includes a proximal portion 150 connected to a distal portion 170 via neck portion 160. Thus, when transducer 130 produces ultrasound energy at the first driving frequencies, transducer 130 induces transverse mechanical vibrations in the distal portion 170 in a direction that is substantially perpendicular to a longitudinal axis extending through a center 164 of applicator 100. Inducing these transverse mechanical vibrations in the distal portion 170 at a time that energy delivery surface 180 of sonotrode 140 is engaged with, or coupled to an upper surface of a biological tissue such as an epidermis (not shown) causes transverse mechanical waves of an ultrasound frequency to be delivered to the biological tissue.

Energy delivery surface 180 of sonotrode 140 may be implemented as a substantially convex surface (e.g., having a hemispherical shape). This shape may be useful for scattering incident ultrasound waves at different angles within the treated biological tissue. In some embodiments, energy delivery surface 180 features one or more ridges and/or protrusions, or alternatively one or more dimples or indentations (not shown, see FIG. 2).

Sonotrode 140 and transducer 130 may be cooled through a cooling apparatus 122, which may feature a cooling device 124 for transducer 130; a cooling pipe 126 for cooling device 124, and a further water connector 128 for receiving water into apparatus 122 (the connection to an external water source is not shown). Water cooling helps to provide a stable level of ultrasound energy output by maintaining stabilization of the resonant frequency and hence supports the below described optional embodiment for determining acoustic pressure by measuring transducer voltage. Of course other types of cooling could alternatively be used with the presently-disclosed apparatus and could easily be selected by one of ordinary skill in the art for use in addition to or in place of water cooling. In some embodiments, cooling is on more than one part of the transducer. In some embodiments, the sonotrode itself is cooled, as well as the transducer.

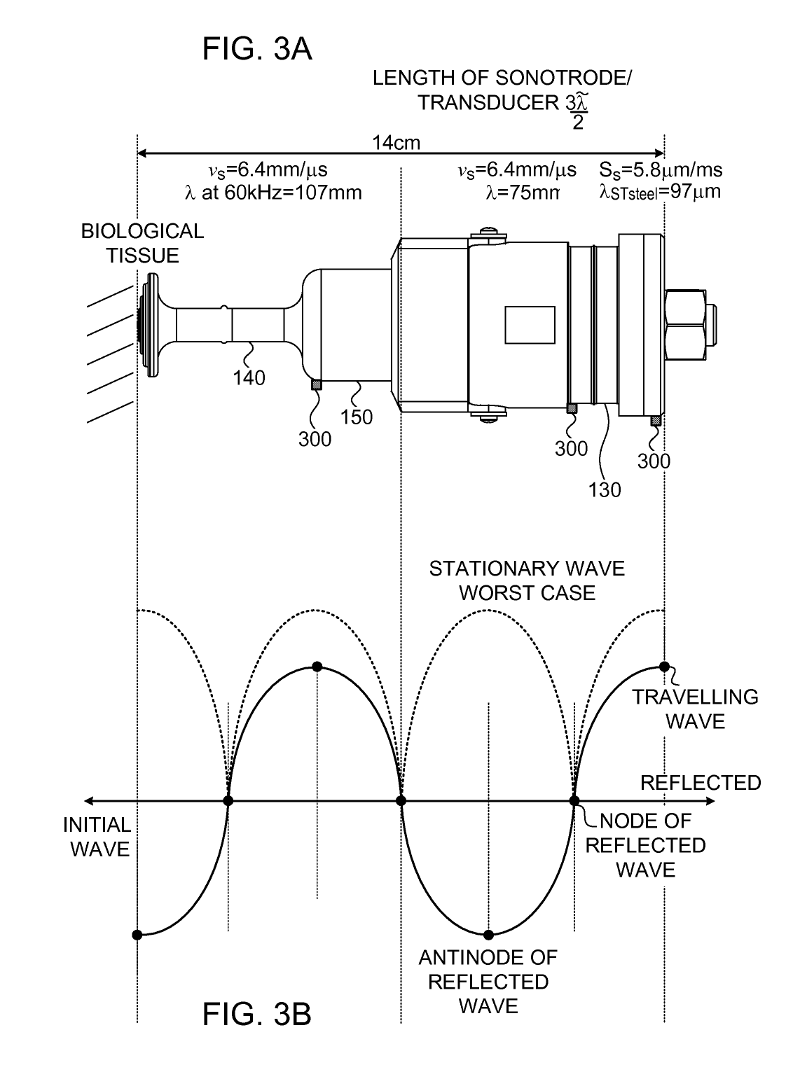

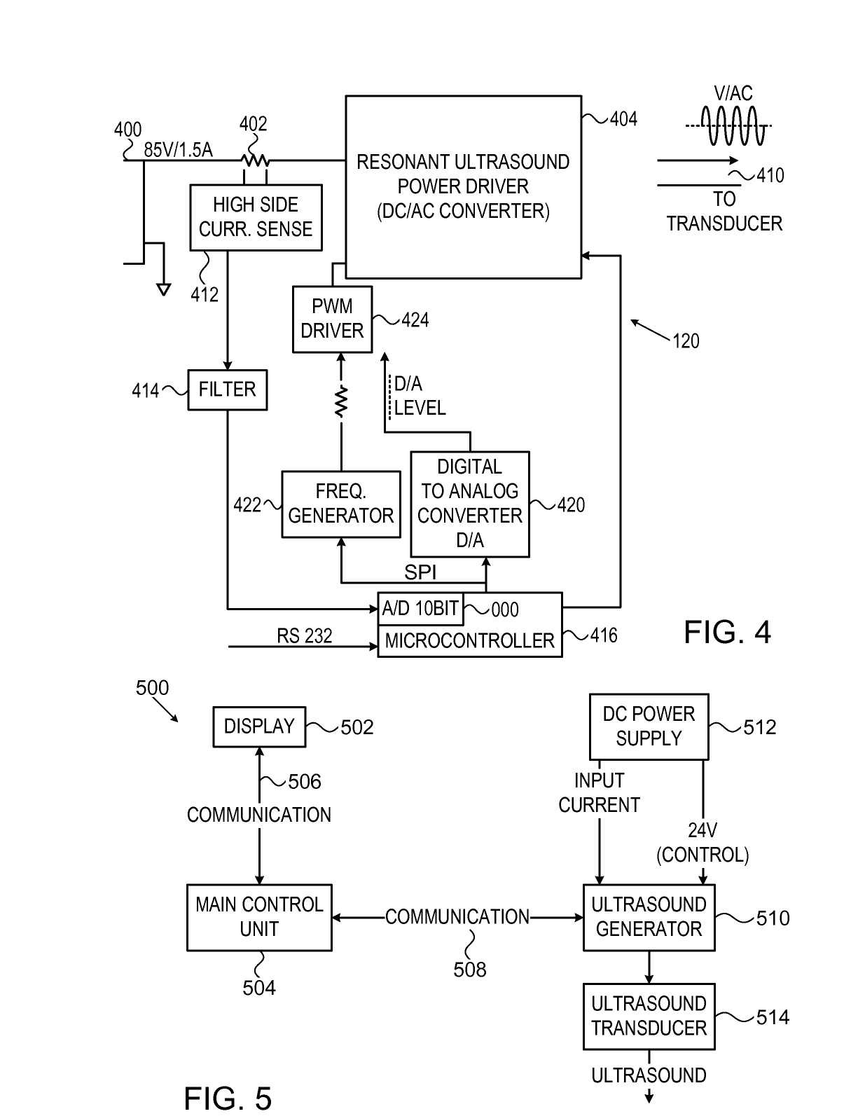

Applicator 100 may also include a device controller 120 for regulating the electrical power delivered to transducer 130 (for example, for controlling the amplitude and/or frequency of transducer 130 and/or for controlling one or more pulse parameters in the event that transducer 130 generates pulsed ultrasound energy). The electrical power may be provided by a power source (not shown). Device controller 120 may suitably be implemented as a card having a plurality of components as described in greater detail below with regard to FIG. 3A, the algorithm of FIG. 3B and the method of treatment of FIG. 4.

It is noted that device controller 120 may be implemented in any combination of electrical circuitry and executable code modules. Although controller 120 is drawn in close proximity of sonotrode 140, this is not a requirement. In some embodiments, controller 120 is optionally attached to and/or associated with a separate console (not shown).

Device controller 120 may communicate with transducer 130. In addition, device controller 120 may communicate with a display 140 for providing feedback (e.g. of an alert signal) to the user.

Device controller 120 may also be in communication with a second display (not shown, see FIG. 5) through a tube (not shown) that is attached to applicator 100 through an adaptor 146.

Even though sonotrode 140 and energy delivery surface 180 are in close contact with an upper surface of the biological tissue, there still may be some atmospheric air layer between the two. For this reason, a material having an intermediate acoustic impedance (for example, a petroleum jelly such as Vaseline®) that is greater than the acoustic impedance of biological tissue but less than the acoustic impedance of the sonotrode may be applied to energy delivery surface 180. The selected material may also be compatible with the operating environment of energy delivery surface 180 and the biological tissue being treated; for example, the amount of heat generated may cause certain acoustic gels to melt and to cease to have “gel-like” properties, whereupon a stiffer gel such as petroleum jelly may be more appropriate.

Furthermore, as discussed below, it is desirable to improve acoustic coupling between sonotrode 140 and biological tissue (i.e., to reduce the amount of reflected power), and a gel such as petroleum jelly may be useful for this purpose as well. When used in this manner, petroleum jelly fills up the voids between the applied sonotrode surface and biological tissue, “replacing” the air, and improving acoustic impedance matching of the system. This may decrease the fraction of ultrasonic power that is reflected.

Applicator 100 may be operated by the user through grasping handle 190 and then moving applicator 100 transversally over the surface of biological tissue (for example, at a minimal speed of 0.5 cm/sec or 1 cm/sec or 2 cm/sec or 3 cm/sec for a minimum distance that is at least 5 cm or 10 cm or 15 cm) as transverse and/or longitudinal ultrasound waves are delivered to biological tissue. The movement of applicator 100 over the treated tissue may be useful for improving energy coupling such as by generating a pressure between energy delivery surface 180 and the tissue.

This may provide a better ultrasound coupling, and is useful for facilitating and ensuring treatment of the entire region sought to be treated. However, it is clearly desirable for the operator to operate applicator 100 in a consistent manner. For example, if the operator uses a repetitive motion to treat the tissue, such as a circular or elliptical motion, then such a motion may cover a similar surface area with each motion and also may feature a similar pressure by the operator. When shear waves are used, consistency is important as a minimum amount of shear waves need to impact the adipocytes for treatment. Shear waves are more useful for treating adipocytes but have low penetration; typically only 10% of the energy penetrates the cells. Thus, it is important for at least a certain amount of shear waves to impact the cells in order for treatment to be effective.

However, different conditions may result in different operators providing dissimilar treatment; indeed, the same operator at different times (or even within a single treatment session, for example as the result of fatigue) may provide different and inconsistent treatment, hence reducing the efficacy and reliability of the treatment results.

To provide motion-dependent feedback, controller 120 features an accelerometer 192, to determine the speed of operator movement of applicator 100. If the motion adopted by the operator is circular, then measurement of radial acceleration provides an indication of angular velocity. The accelerometer is may implemented as a MEMS (micro-electro-mechanical sensor) which are current widely available and used widely in smart phones and games controllers. MEMS sensors are also available to measure inclination and to measure linear velocity and any such sensor may be used in embodiments of the invention to provide motion feedback to the operator. This is not to exclude other forms of sensors, such as gyroscopes, that can be used to determine the inclination of the applicator.

Controller 120 may be implemented as ‘electronic circuitry.’ Controller 120 also may feature the necessary software and/or firmware, and also optionally features a processor of some type (which may for example comprise a logic gate) as described in greater detail below with regard to FIG. 3A.

In some embodiments, controller 120 is operative to determine acoustic pressure applied to the tissue being treated, which is dictated by the amount of acoustic power produced by the transducer and the effectiveness of the coupling between energy delivery surface 180 and the biological tissue, the latter being itself dependent upon the pressure applied by the operator through handle 190.

Acoustic pressure may be determined according to the effect of distorting of resonant curves of the electrical resonance by the extent of acoustic load. The treated tissue contact decreases the reflected acoustic energy and consequently the standing wave amplitude; the voltage amplitude at the electrical resonant frequency also decreases, as described in greater detail below with regard to FIG. 3b. On the other hand, poor acoustic coupling between the energy delivery surface 180 and the tissue will result in higher levels of reflection of the acoustic energy.

Optionally, the ultrasound treatment system comprises a coupling sensor element detecting a non-efficient coupling through a change of acoustic standing wave parameters. The standing wave ratio (or swr) depends on acoustic coupling, increasing at insufficient coupling conditions (higher reflections). Changes in the standing wave can be detected by measuring the waves at or near an antinodal point.

The acoustic coupling detecting system can also include or may be based on variations of electrical parameters of the transducer, namely change of voltage drop, transducer current, resonant conditions of the transducer (if resonant feeding of the transducer is applied). Such resonant conditions may optionally comprise one or more of drift of the resonant frequency of the electrical circuit itself, resonant current and voltage.

For example, acoustic coupling factors are reflected in the AC-voltage supplied to transducer 130 and also in the DC-current of the transducer driver (not shown); as acoustic coupling increases and hence acoustic pressure on the biological tissue increases, the AC-voltage decreases, as does the DC-current, in a correlated manner. Therefore, measurement of the voltage may optionally be used to determine acoustic pressure.

One or more factors regarding the performance of the operator, for example related to speed of movement of applicator 100 and/or acoustic pressure, may be analyzed by controller 120. Feedback regarding the analysis of such factors, for example with regard to whether the operator is performing optimally or whether one or more changes are required, may be indicated to the operator, for example through display 140. Display 140 may optionally comprise a visual display, an audio display, a tactile feedback display and so forth. For example and without limitation, as a visual display, display 140 may optionally comprise one or more LED lights to provide operator feedback.

FIG. 2A shows sonotrode 140 and transducer 130 in a cross-sectional view. All components having the same or similar function as in FIG. 1 have the same reference numbers; FIGS. 2B-2E relate to various aspects of acoustic coupling according to at least some embodiments of the present invention.

Energy delivery surface 180 features a plurality of indentations 200 as shown, for better coupling with the biological tissue.

As shown, physical coupling between transducer 130 and sonotrode 140 is may be provided through a screw 202 and a threaded hole 204 for receiving screw 202.

Sonotrode 140 and transducer 130 are held in an appropriate orientation through a flange 194 and a nut 196, also shown in FIG. 1.

In this non-limiting example, sonotrode 140 is symmetric about longitudinal axis 164, though this is not a limitation; a sonotrode according to the invention may be asymmetric about the longitudinal axis 164.

As shown, sonotrode 140 is constructed, for example, as a solid and/or hollow form, such that when ultrasound transducer 130 generates longitudinal mechanical waves of a particular driving ultrasound frequency within proximal portion 150, energy of these longitudinal waves travels into neck portion 160 and induces distal portion 170 to vibrate at an ultrasound frequency in a direction that is substantially perpendicular to the longitudinal direction of the sonotrode (i.e., a direction parallel to longitudinal axis 164). Thus, ultrasound transducer 130 may induce a standing wave in distal portion 170 in a direction that is substantially perpendicular (e.g., within a tolerance of 25, 20, 10, or 5 degrees) to longitudinal axis 164.

Thus, sonotrode 140 is operative to “convert” plunger-type vibrations in proximal portion 150 and neck portion 160 into bending-type (or transverse) vibrations in distal portion 170. Sonotrode 140 may be dimensioned so that: (i) the ratio between dimension B of the neck portion 160 parallel to the elongate axis of the neck and dimension d1 of the neck portion 160 perpendicular to the elongate axis of the neck is at least 1.5 (or at least 2 or at least 2.5); (ii) the ratio between a dimension d2 of the distal portion 170 perpendicular to the elongate axis of the neck and dimension B of the distal portion 170 parallel to the elongate axis of the neck is at least 2 (or at least 2.5 or at least 3); (iii) the ratio between dimension D of the proximal portion 150 perpendicular to the elongate axis of the neck and dimension d1 of the neck portion 160 perpendicular to the elongate axis of the neck is at least 2.5 (or at least 3 or at least 3.5); (iv) the ratio between dimension d2 of the distal portion 170 perpendicular to the elongate axis of the neck and dimension d1 of the neck portion 160 perpendicular to the elongate axis of the neck is at least 2 (or at least 2.5 or at least 3).

An acoustic sensor 198 is shown, which in this optional non-limiting example measures the voltage drop across the transducer.

FIGS. 2B-2C show the effect of acoustic coupling on electric resonance, for demonstrating a method according to at least some embodiments of the present invention for determining coupling according to measurements of electrical resonance of the electrical circuit that includes the transducer. This method is applicable for different types of ultrasound waves.

If the sonotrode material features aluminum and the treated tissue is human skin, then for longitudinal waves the reflection coefficient is 68% approximately. Therefore, 32% of acoustic energy will be coupled and will be transmitted to the treated biological tissue and 68% of energy will be reflected backwards. If the sonotrode has poor coupling and transmits the acoustic energy to air, the value of reflected energy will reach 95-98%.

For typical “real life” treatment of a biological tissue such as skin, the amount of acoustic coupling will be between these two numbers. The situation is more complicated for sonotrodes such as that shown in FIG. 2A, featuring “dimples” or protrusions, and which are used for “dynamic” or motion based treatment (as described herein for at least some embodiments of the present invention). However, generally the presence of poor coupling can be detected in these situations as well.

As shown in FIG. 2B, for poor acoustic coupling, the voltage at the transducer increases, while for good acoustic coupling, the voltage at the transducer decreases, as measured for example by sensor 198. FIG. 2C shows the complete electrical circuit with the transducer, indicating that the resonance of the circuit itself may also optionally be measured. FIG. 2C also features an equation which may be explained as follows. The transducer can be characterized by certain capacitance C. If resonant inductance L is turned in series or in parallel with this transducer capacitance, the circuit will be resonant at certain frequency. The resonant frequency f=½π√LC. The Q-factor characterizes an efficiency of the circuitry.

The electrical resonant frequency should correspond with acoustic resonance of the system. The electrical resonance depends on inductor (L) tuning. With proper inductor it is possible to achieve a pure sine voltage (with 25-30 dB of harmonics) and pure acoustic resonance of the transducer/sonotrode assembly. The Q-factor of the acoustic resonator (sonotrode/transducer) is dependent on the load of the resonance structure. The acoustic resonance is reachable if the length of the assembly is equal to the whole number of half-wavelength L=nλ/2. (in the illustrated case the length of sonotrode is approximately equal to 3 half-wavelengths. If the acoustic resonant structure is unloaded (full reflection case—operation with air), the standing wave reaches to its maximum and acoustic amplitude in anti-nodes will be maximal. In its turn the electrical resonance will have highest Q-factor, and HF-voltage amplitude will be highest. If the acoustic resonator is in good contact with biological tissue, the energy losses from the resonator will be higher as more useful energy is transferred to the tissue; it will be equivalent to decreasing of Q-factor of acoustic resonance. Simultaneously, the electrical resonance voltage will be decreases. Upon calibration procedure, it is possible to make certain criteria and indicate if the voltage decrease in critical and correction procedures will be implemented or situation is acceptable.

FIG. 2D shows poor contact, while FIG. 2E shows good contact of the sonotrode with biological tissue. The coupling material, such as petroleum jelly, may fill the cavities between sonotrode surface and biological tissue, eliminating or reducing any presence of air and improve acoustic coupling.

FIGS. 3A and 3B show the effect of good acoustic coupling on voltage, for demonstrating a method for determining coupling according to measurements of parameters (amplitude) of standing wave. This method is more effective for longitudinal ultrasound waves. Implementation of the method with shear wave ultrasound is less sensitive but still possible.

As previously described, according to at least some embodiments the ultrasound system is capable of operating alternately in both longitudinal and transverse modes, one mode being of resonance being excited at an operating frequency of 60 KHz and the other at 70 KHz. The longitudinal mode, which is typically used for 10% to 25% of the operating time, may be used to detect coupling through standing wave change. Even if treatment is not effected using longitudinal waves, optionally such waves may be transmitted specifically to detect acoustic coupling.

As shown in FIG. 3A, the device of FIG. 2A optionally features one or more acoustic sensors 300 (FIG. 3A shows a simplified version of the device of FIG. 2A for clarity). Optionally, the acoustic coupling sensors may detect acoustic power placed on the area of antinodes of ultrasound standing wave, as shown with regard to FIG. 3B. For this optional embodiment, the calibration process will include detecting of reflected wave conditions under proper coupling and with “air” (no proper coupling); during treatment, the method optionally features measurements of the reflected power amplitude during operation of the device. If the difference of two signals will be more than certain threshold, since at the antinodes clearly the amplitude is higher with poor coupling, the feedback system will provide an operator (human or automatic) with a signal to increase coupling, for example by increasing pressure and/or altering the angle of pressure.

Turning now to FIG. 4, controller 120 and the corresponding electrical circuit for the transducer is shown in more detail. As shown, an input connector 400 receives current, which is then split by a current shunt 402. DC input from shunt 402 is converted by a resonant ultrasound power driver (DC/AC converter) 404 to AC output to a transducer connector 410 (the transducer itself is not shown).

Current shunt 402 also provides current to a current sensor 412 and then to a filter 414, after which it is provided to a microcontroller 416 as a non-limiting example of a controller. Microcontroller 416 determines the resonance frequency of the resonant ultrasound power driver 404. Also, microcontroller 416 outputs to a digital to analog (D/A) converter 420 and to a frequency generator with a sawtooth output 422, which in turn communicate with a PWM driver 424.

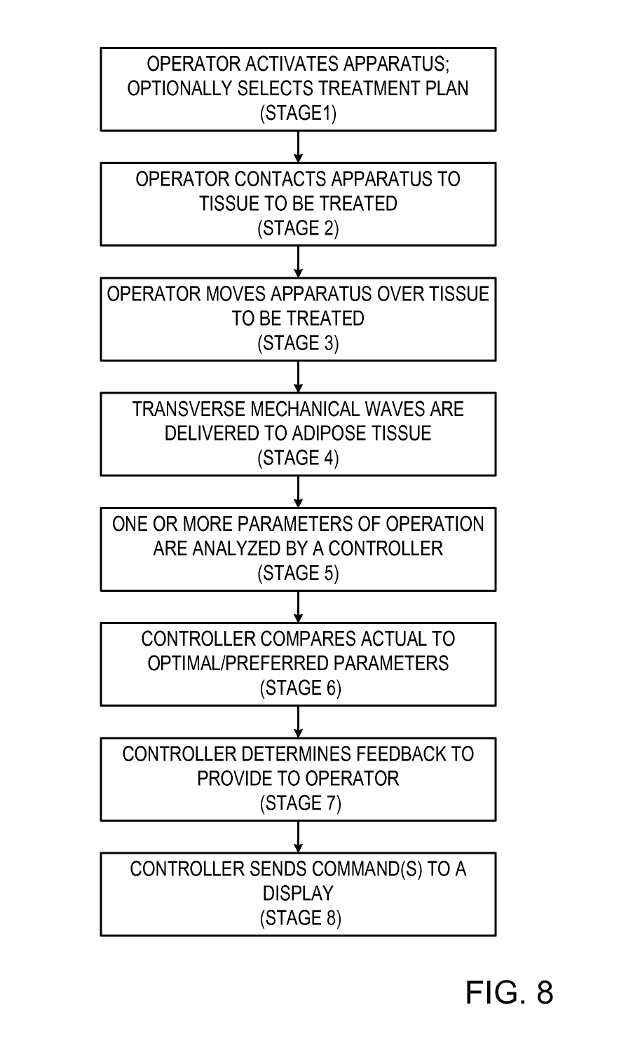

FIG. 5 shows an exemplary ultrasound system 500 featuring a display 502 for providing feedback to the operator, which for this embodiment may be a human operator. Display 502 may optionally be any type of display (audio, visual, tactile, or a combination thereof). Display 502 may be in communication with a main control unit 504 through a communication channel 506 as shown. Main control unit 504 is may also be in communication with an ultrasound generator 510 through a second communication channel 508. A power supply 512 provides power to ultrasound generator 510, which in turn supplies power to an ultrasound transducer 514. Operation of system 500 is described in greater detail with regard to the method of FIG. 8 below.

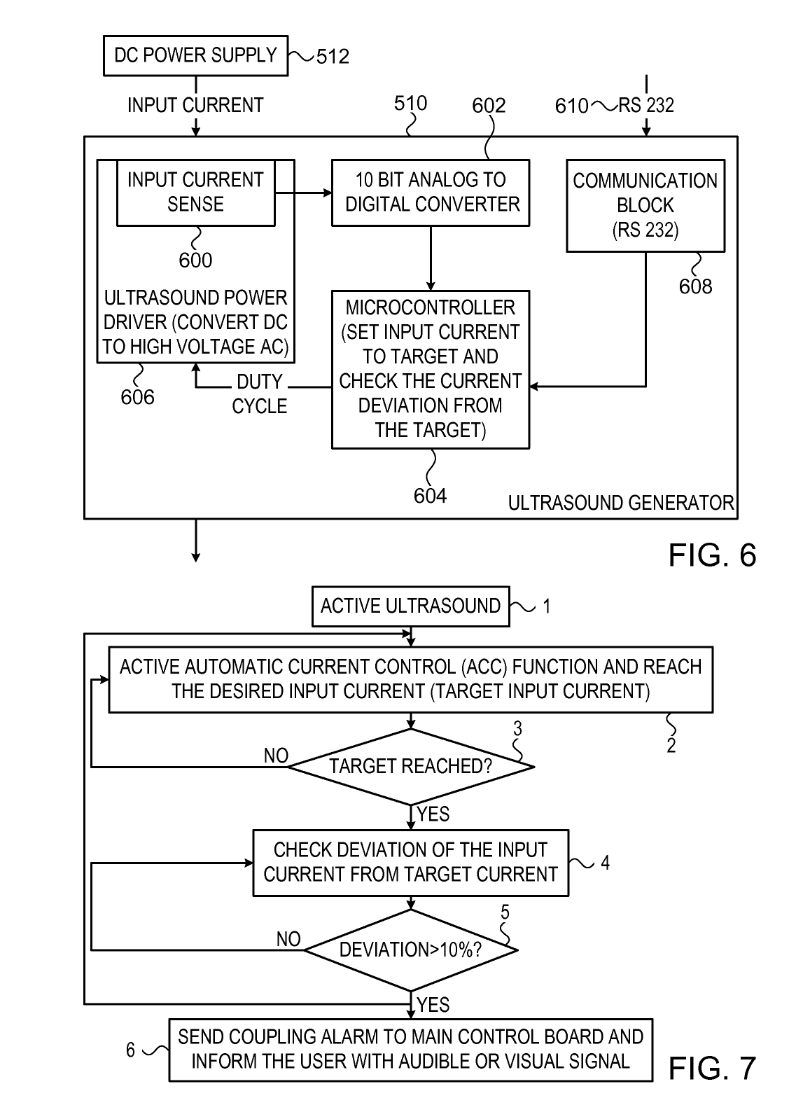

FIG. 6 is a schematic block diagram of a non-limiting embodiment of ultrasound generator 510, also showing power supply 512. As shown, ultrasound generator 510 optionally features an input current sensor 600, which senses incoming current and through an analog to digital converter 602, provides information regarding the incoming current to a microcontroller 604. The incoming current also drives an ultrasound driver 606, which is controlled by microcontroller 604. Communication with ultrasound generator 510 is possible through an optional communication block 608 in communication with microcontroller 604, which in turn connects externally to ultrasound generator 510 through a connector 610, which may for example optionally be a RS232 connector as shown.

FIG. 7 shows a flowchart of an exemplary, optional method for determining a level of acoustic coupling. In stage 1, the ultrasound system is activated. In stage 2, an automatic current control function is activated; periodically it checks in stage 3 whether the desired input current has been reached. If not, the method returns to stage 2, until the desired input current has been reached. Once it has been reached, in stage 4 the process of checking during operation of the system is performed. If the deviation is less than some upper bound in stage 5, then the method returns to stage 4, which may be performed periodically during operation of the ultrasound system. Otherwise, in stage 6 feedback is provided to the operator, for example through an alarm or some type of display.

FIG. 8 is a flowchart of an exemplary, illustrative non-limiting method for treating adipose tissue with transverse ultrasound waves. In stage 1, the operator activates the apparatus and optionally also selects one or more treatment plans, such that feedback as described below is optionally provided with regard to the selected treatment plan. For example and without limitation, the operator may optionally input information regarding one or more patient details, information about an area or areas of the body to be treated; and so forth.

In stage 2, the operator contacts the apparatus (and more specifically the energy delivery surface) to the biological tissue to be treated, which is described herein with regard to skin for the purpose of illustration only and without any intention of being limiting. In stage 3, the operator moves the apparatus over the tissue to be treated.

In stage 4, the transverse mechanical waves of ultrasound frequency are delivered to adipose tissue beneath the dermis—for example, using a sonotrode such as or similar to the sonotrode depicted in FIGS. 1 and 2.

In stage 5, one or more parameters of the operation of the apparatus by the operator are analyzed by a controller, which may for example optionally be the controller of FIGS. 1 and 4. For example, the one or more parameters of the operation may optionally include acoustic pressure, acceleration and/or apparatus orientation.

In stage 6, the controller compares the one or more parameters of actual operation to data regarding optimal or preferred parameters of operation. In stage 7, the controller determines feedback to be provided to the operator regarding this comparison. In stage 8, the controller sends one or more commands to a display to provide such feedback to the operator.

1-15. (canceled)

16. A treatment apparatus for applying mechanical ultrasonic vibrations to an area of tissue, comprising an applicator movable by an operator over the area of tissue to be treated, the applicator having mounted therein an ultrasonic transducer for generating mechanical vibrations and a sonotrode for transmitting the vibrations generated by the transducer to the tissue to be treated, wherein the transducer is operative to apply two different frequencies alternately to the sonotrode so as to cause the sonotrode to vibrate alternately in a longitudinal mode and in a transverse mode, the apparatus further comprising a sensor for measuring, at least during operation in the longitudinal mode, an operating parameter indicative of the efficiency of the acoustic coupling between the sonotrode and the tissue to be treated, and electronic circuitry for generating a signal when the efficiency of the acoustic coupling drops below a predetermined threshold.

17. The treatment apparatus of claim 16, wherein the sensor is responsive to vibrations reflected at an interface between the sonotrode and the tissue to be treated during operation in the longitudinal mode, the reflected vibrations setting up a standing wave in the sonotrode of which the amplitude decreases with increase in coupling efficiency.

18. The treatment apparatus of claim 17, wherein the sensor comprises at least one vibration sensor in contact with a region of the sonotrode close to an internode of the standing wave set up by the vibrations reflected at the interface between the sonotrode and the tissue to be treated.

19. The treatment apparatus of claim 16 wherein the signal is an alert signal.

20. The treatment apparatus of claim 19 wherein the alert signal is a visual alert signal.

21. The treatment apparatus of claim 19 wherein the alert signal is an audio alert signal.

22. An ultrasound treatment method comprising:
operating an ultrasound device to transmit ultrasound vibrations including transverse ultrasound vibrations to biological tissue via a device sonotrode in contact therewith;
monitoring an ultrasound device operating parameter that is descriptive of a coupling efficiency between the sonotrode and the biological tissue; and
contingent upon the monitored coupling efficiency being below a threshold value, generating a coupling alert signal.

23. The ultrasound treatment method of claim 22 wherein:
i. the ultrasound device cycles between longitudinal and transverse operating modes; and
ii. when the ultrasound device is in the transverse mode, the generated coupling alert signal is based primarily upon longitudinal-mode measurements of the operating parameter.

24. The ultrasound treatment method of claim 23 wherein a frequency of the device cycle is at least 0.2 Hz.

25. The ultrasound treatment method of claim 23 wherein a frequency of the device cycle is at least 0.4 Hz.

26. The ultrasound treatment method of claim 23 wherein the sonotrode vibrates in the transverse mode for a substantial majority of each of a plurality of the device cycles.

27. The ultrasound treatment method of claim 26 wherein a frequency of the device cycle is at least 0.2 Hz.

28. The ultrasound treatment method of claim 27 wherein a frequency of the device cycle is at least 0.4 Hz.

29. The ultrasound treatment method of claim 22 wherein the operating parameter is monitored by sensing a strength of vibrations reflected at an interface between the sonotrode and the biological tissue to be treated.

30. The ultrasound treatment method of claim 22 wherein ultrasound energy of the ultrasound device is generated by a transducer powered by an oscillatory drive circuit, and wherein the operating parameter is monitored by monitoring a voltage across the drive circuit and/or by monitoring a current drawn therefrom.

31. The ultrasound treatment method of claim 22 wherein ultrasound energy of the ultrasound device is generated by a transducer powered by an oscillatory drive circuit, and wherein the monitored operating parameter descriptive of the coupling efficiency is a Q-factor thereof.

32. The ultrasound treatment method of claim 22 which further comprises sensing a speed or acceleration of the sonotrode as it moves along a surface of the biological tissue and generating a motion alert signal when the speed of acceleration lie outside a desired range

33. The ultrasound treatment method of claim 22 which further comprises sensing a tilt of the sonotrode relative to a surface of the biological tissue and generating an inclination alert signal when the inclination is outside a desired range.

34. An ultrasound treatment device for treating biological tissue, the device comprising:
a. a sonotrode;
b. an ultrasound transducer coupled to the sonotrode and configured to induce mechanical vibrations of an ultrasound frequency therein so as to deliver ultrasound energy to the biological tissue via an energy delivery surface of the treatment device facing and coupled to the biological tissue; and
c. electronic circuitry operative to monitor an ultrasound device operating parameter that is descriptive of a coupling efficiency between the sonotrode and the biological tissue and to generate, contingent upon the monitored coupling efficiency being below a threshold value, a coupling alert signal.

35. The ultrasound treatment device of claim 34 wherein:
i. the ultrasound device is operative to cycle between a longitudinal mode and transverse modes; and
ii. the electronic circuitry is operative so that when the device is in the transverse mode, the generated coupling alert signal is based primarily upon longitudinal-mode measurements of the operating parameter.

36-43. (canceled)
